(19) United States
(12) Patent Application Publication
(10) Pub. No.: US 2024/0310262 A1
(43) Pub. Date: Sep. 19, 2024

(54) METHOD FOR MEASURING OIL VISCOSITY AND VISCOSITY TESTING KIT FOR PERFORMING THE METHOD

(71) Applicant: AKTIEBOLAGET SKF, GOTEBORG (SE)

(72) Inventors: Nicola DE LAURENTIS, Houten (NL); Pieter Martin LUGT, Vianen (NL); Laurine YOE, Gemert (NL)

(21) Appl. No.: 18/599,303

(22) Filed: Mar. 8, 2024

(30) Foreign Application Priority Data
Mar. 15, 2023 (DE) 102023202323.7

(51) Int. Cl. G01N 11/00; G01N 33/28

(52) U.S. Cl. CPC G01N 11/00; G01N 33/28

(57) ABSTRACT

A method for measuring a viscosity of an oil includes placing a drop of the oil on a piece of blotter paper to form a stain, measuring a size of the stain at a given time after placing the drop of the oil on the piece of blotter paper and determining the viscosity of the oil by comparing the size of the stain with empirical data relating stain size, time and oil viscosity. Also a kit for performing the method.

### CROSS-REFERENCE

This application claims priority to German patent application no. 10 2023 202 323.7 filed on Mar. 15, 2023, the contents of which are fully incorporated herein by reference.

### TECHNOLOGICAL FIELD

The present disclosure is directed to material testing, and more particularly to devices and methods for testing properties of oils used in machine lubrication and even more specifically to devices and methods for testing oil viscosity.

### BACKGROUND

When selecting the right oil for a target application, viscosity is generally considered to be one of the most important properties of a lubricating oil and is always present in a technical data sheet of the lubricating oil. However, the viscosity of an oil can change over time and as the oil is exposed to the conditions of use.

Measuring the viscosity of worn-out oils is commonly done as part of an oil analysis and diagnosis service over the lifetime of the used oils. A viscosity measurement can offer information on the degradation status of the oil as it is related to oxidation, contamination, and to the health of the machine in which it is employed. Thus, it can be used as a condition monitoring tool.

Oil viscosity can be measured in many ways. Usually, it is measured by using capillary or rotational viscometers. However, viscometers are expensive and impractical in certain industrial environments. A typical sample volume for a viscosity measurement with a viscometer amounts to 5 ml. Although relatively small, this amount is not always available.

Furthermore, in standard viscometers, care must be taken to ensure high cleanliness of the instrumentation, via the use of solvents, and to also avoid the presence of air bubbles in the oil, which can both greatly affect the results and provide an erroneous reading. A calibration of the viscometers must also be done.

Therefore, a typical practice is to send an oil sample to off-site laboratories for viscosity measurements. This can be both costly and slow.

### SUMMARY

There is thus a need to provide a viscosity measurement method that can be more easily implemented.

One aspect of the present disclosure is to provide a method for measuring the viscosity of an oil, which method includes: placing a drop of oil on a blotter paper to form a stain, measuring growth of the stain over a scale of time, and determining the viscosity of the oil by comparing the growth of the stain with predetermined master curves.

Thus, this method is simple and accessible to everyone. It is also inexpensive, fast, does not involve the use of solvents and requires a very small amount of oil.

In one embodiment, the method further includes the following steps before measuring the growth of the stain: placing the blotter paper on which the stain is formed onto a temperature-controlled plate and setting the temperature of the plate at a temperature within a predetermined temperature range.

Advantageously, the temperature-controlled plate is set at a temperature between 50° C. and 60° C., preferably between 53° C. and 57° C., and more preferably to a temperature of 55° C.

Advantageously, the drop of oil has a predetermined volume set between 50 and 100 μL, preferably of 80 μL, which is preferably obtained by using an appropriately sized pipette.

In an embodiment, the growth of the stain is measured with a scale printed on the blotter paper.

Advantageously, the drop of oil is placed on the blotter paper so that it forms a circular stain of at least 10 mm diameter.

In an embodiment the viscosity of the oil is determined with a maximum deviation of 14% as compared to the actual viscosity of the oil.

Advantageously, the master curves are determined by measuring the growth of the stain of oils whose viscosity is known.

One other aspect of the disclosure is to provide a viscosity testing kit for measuring the viscosity of an oil implementing the method as previously defined. The kit includes a pipette, a temperature-controlled plate, and at least one sheet of blotter paper.

Advantageously, a scale is provided on at least one blotter paper.

### BRIEF DESCRIPTION OF THE DRAWINGS

Other advantages and features of the disclosure will appear from the following detailed description of an embodiment of the invention, which is a non-limiting example, illustrated in the appended drawings of which:

FIG. 1 is a schematic representation of the steps of the method according to an embodiment of the disclosure.

FIG. 2 is a schematic representation of a viscosity testing kit according to an embodiment of the disclosure.

FIG. 3A, FIG. 3B and FIG. 3C are schematic views of a blotter paper showing the changing size of an oil stain over time.

FIG. 4 is a graph showing master curves that represent the growth of an oil stain over time for several types of oil each having a known viscosity.

### DETAILED DESCRIPTION

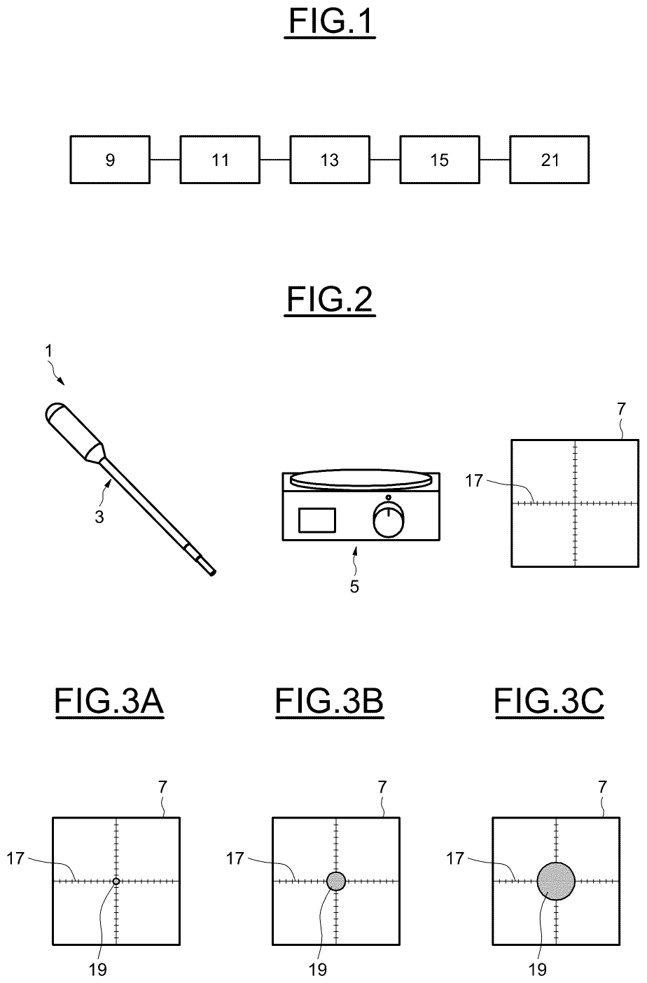

The FIG. 1 shows a plurality of steps of a method for measuring the viscosity of an oil. The oil may be for example a PAO (Polyalphaolefin) oil used a lubricant.

In order to measure the viscosity of the oil, a viscosity testing kit 1 as depicted in FIG. 2 may be used. The viscosity testing kit 1 comprises a pipette 3, a temperature-controlled plate 5 and at least one blotter paper 7.

The method comprises a first step 9 of placing a drop of oil on the blotter paper 7 so that it forms a stain. The stain formed by the drop preferably has a circular shape and is preferably placed in the center of the blotter paper 7, which provides room for the stain to grow.

The stain also preferably has a shape of at least 10 mm diameter in order to distinguish it clearly. However, the diameter of the stain must not be greater than one half the diameter or width of the blotter paper 7.

In order to form a suitable stain, the drop of oil should have a predetermined volume of from 50 μL to 100 μL, preferably of 80 μL.

Advantageously, the predetermined volume can be set by using a pipette 3, for example the pipette 3 of the viscosity testing kit 1, with which the drop is placed on the blotter paper 7.

Preferably, the method comprises a step 11 of placing the blotter paper 7 on which the stain is formed on the temperature-controlled plate 5. Alternatively, the step 11 of placing the blotter paper 7 on the plate 5 can be realized before the step 9 of placing a drop of oil on the blotter paper 7.

A step 13 of setting the temperature of the plate 5 is then performed. The temperature is set at a temperature that falls within a predetermined range of temperature.

Alternatively, this step 13 of setting the temperature of the plate 5 can be performed before the step 11 of placing the blotter paper 7 on the plate 5, or more preferably before the drop of oil is placed on the blotter paper 7. The plate 5 should be turned on and set with a stabilized temperature before placing the oil drop on the paper or before placing the blotter paper 7 on the plate 5.

Advantageously, the temperature-controlled plate 5 is set at a temperature between 50° C. and 60° C., preferably between 53° C. and 57° C., and more preferably at 55° C.

This allows an operator to implement the present method with a precise temperature, in order to be able to reproduce the method and have comparable values between different oils and/or between different samples of the same oil.

Then, the method comprises a step 15 of measuring/observing the growth of the stain over a period of time. More precisely, this step 15 comprises the measure of the diameter of the stain periodically over a period of time, for example every five minutes over the course of two hours.

Advantageously, a scale 17 is printed on the blotter paper 7 in order to facilitate the measure of the size of the stain. More precisely, two mutually perpendicular scales 17 are printed on the blotter paper.

FIGS. 3A, 3B and 3C show different a blotter paper 7 on which an oil stain 19 has been formed at different points in time.

FIG. 3A shows the blotter paper 7 just after a drop of oil has been thereon. A scale 17 is printed or otherwise marked on the blotter paper 7.

FIG. 3B shows the blotter paper 7 of FIG. 3A five minutes after the drop of oil was been placed on the blotter paper 7. The diameter of the stain in FIG. 3B is about twice as large as the diameter of the stain in FIG. 3A.

FIG. 3C shows the blotter paper 7 of FIG. 3A about sixty minutes after the drop of oil was placed on the blotter paper. The diameter of stain is about twice as large as the diameter of the stain of FIG. 3B.

The correlation between the size of the stain 19 and the time after having placed the drop on the blotter paper 7 depends on the oil used, and its viscosity and on how worn out the oil is.

After having measured the growth of the stain over time, the method comprises a step 21 of determining the viscosity of the oil of the stain 19 by comparison of the previous measures with predetermined master curves.

The master curves are determined using the same protocol of measuring the growth of a stain 19 of oil, but for oils whose viscosity is known.

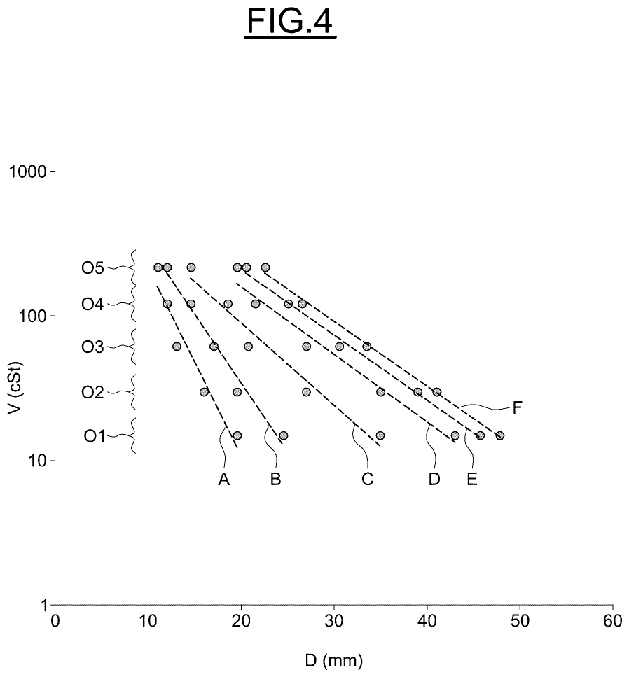

FIG. 4 is a representation of master curves of five different PAO (Polyalphaolefin) oils O1, O2, O3, O4 and O5, each oil having a different viscosity V, measured in centistokes. Six measures A, B, C, D, E and F for each of the oils O1, O2, O3, O4 and O5 are represented on FIG. 4, respectively made 5 minutes, 10 minutes, 30 minutes, 60 minutes, 90 minutes and 120 minutes after having the drop of oil was placed on the blotter paper 7. The measured diameter D of the drop at each of these times for each of these oils is shown by a dot on the graph of FIG. 4. The blotter papers used to produce this graph were all maintained at a predetermined temperature as discussed above.

Different types of oils can be studied, for example the previous PAO oils, but also mineral oils and ester-based oils.

By comparing measurements of a stain 19, at a given time, of an oil whose viscosity is unknown with these master curves, it is possible to determine the viscosity of the oil.

In addition, the viscosity of a known oil can be measured to see whether it deviates from an expected value. That is, after an oil has been used for an extended period of time, the viscosity of the oil may have decreased to the point that the oil is no longer suitable for use. From FIG. 4, it is expected that a drop of oil O3 should have a diameter of about 29 mm 60 minute after being placed on a piece of blotter paper 7 (with a controlled temperature as discussed above). If the drop of oil O3 instead has a diameter of about 35 mm 60 minutes after it is placed on the blotter paper, it can be determined that its viscosity has dropped significantly and in fact is now similar to the known viscosity of oil O2 which also is expected to produce a stain having a diameter of about 35 mm 60 minute after the drop is placed on the blotter paper.

More precisely, the viscosity of the oil is determined with a maximum deviation of 14% compared to reality.

Representative, non-limiting examples of the present invention were described above in detail with reference to the attached drawings. This detailed description is merely intended to teach a person of skill in the art further details for practicing preferred aspects of the present teachings and is not intended to limit the scope of the invention. Furthermore, each of the additional features and teachings disclosed above may be utilized separately or in conjunction with other features and teachings to provide improved methods and kits for determining a viscosity of an oil.

Moreover, combinations of features and steps disclosed in the above detailed description may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

All features disclosed in the description and/or the claims are intended to be disclosed separately and independently from each other for the purpose of original written disclosure, as well as for the purpose of restricting the claimed subject matter, independent of the compositions of the features in the embodiments and/or the claims. In addition, all value ranges or indications of groups of entities are intended to disclose every possible intermediate value or intermediate entity for the purpose of original written disclosure, as well as for the purpose of restricting the claimed subject matter.

What is claimed is:

1. A method for measuring a viscosity of an oil, comprising:
placing a drop of the oil on a piece of blotter paper to form a stain;
measuring a size of the stain at a given time after placing the drop of the oil on the piece of blotter paper; and
determining the viscosity of the oil by comparing the size of the stain at the given time with empirical data relating stain size, time and oil viscosity.

2. The method according to claim 1, further comprising, before measuring the size of the stain:
placing the piece of blotter paper on a temperature-controlled plate; and
setting a temperature of the plate to a predetermined temperature.

3. The method according to claim 2,
wherein placing the piece of blotter paper on the temperature-controlled plate occurs after placing the drop of the oil on the piece of blotter paper.

4. The method according to claim 2,
wherein the predetermined temperature is between 50° C. and 60° C.

5. The method according to claim 2,
wherein the predetermined temperature is between 53° C. and 57° C.

6. The method according to claim 2,
wherein the predetermined temperature is 55° C.

7. The method according to claim 2,
wherein a volume of the drop of the oil is between 50 μL and 100 μL.

8. The method according to claim 2,
wherein a volume of the drop of the oil is 80 μL.

9. The method according to claim 2,
wherein the piece of blotter paper includes a printed scale; and
wherein measuring the size of the stain comprises measuring the size of the stain against the printed scale.

10. The method according to claim 2,
wherein a volume of the drop of oil and the blotter paper are selected such that the drop of oil produces a stain on the blotter paper having a diameter of at least 10 mm two seconds after the drop of oil is placed on the blotter paper.

11. The method according to claim 2,
wherein the viscosity of the oil is determined with a maximum deviation of 14% compared to reality.

12. The method according to claim 3,
wherein the predetermined temperature is between 55° C.,
wherein a volume of the drop of the oil is 80 μL, and
wherein the blotter paper are selected such that the drop of oil produces a stain on the blotter paper having a diameter of at least 10 mm two seconds after the drop of oil is placed on the blotter paper.

13. The method according to claim 2,
wherein placing the drop of the oil on the piece of blotter paper comprises placing the drop of oil on the piece of blotter paper at an intersection of two mutually perpendicular scales printed on the piece of blotter paper.

14. A viscosity testing kit for measuring a viscosity of an oil, comprising:
a pipette;
a temperature-controlled plate,
at least one piece of blotter paper, and
printed empirical data relating stain size, time and oil viscosity.

15. The viscosity testing kit according to claim 14,
wherein a scale is printed on the at least one piece of blotter paper.

16. The viscosity testing kit according to claim 15,
including instructions for determining the viscosity of an oil in the pipette from a size of a stain created by a drop of the oil on the at least one piece of blotter paper at a given time after placement on the at least one piece of the blotter paper and the printed empirical data.
